(19) United States
(12) Patent Application Publication
(10) Pub. No.: US 2006/0189554 A1
(43) Pub. Date: Aug. 24, 2006

(54) Nanoparticle-Based vaccine delivery system containing adjuvant

(76) Inventors: Russell Mumper, Lexington, KY (US); Zhengrong Cui, Lexington, KY (US)

Correspondence Address: MCDERMOTT WILL & EMERY LLP, 600 13TH STREET, N.W., WASHINGTON, DC, 20005-3096, US

(21) Appl. No.: 10/528,817

(22) PCT Filed: Sep. 24, 2003
(86) PCT No.: PCT/US03/29536

§ 371 Date: Mar. 7, 2006

Related U.S. Application Data
(60) Provisional application No. 60/412,780, filed on Sep. 24, 2002.

(51) Int. Cl. A61K 48/00; A61K 9/14

(52) U.S. Cl. 514/44; 424/489; 977/916

(57) ABSTRACT

A vaccine delivery system comprising adjuvant and nanoparticles comprising an immunogenic agent is provided. A method of immunizing an animal comprising administering a nanoparticle-based vaccine delivery system is also provided.

This application claims priority to provisional application Ser. No. 60/412,780, filed Sep. 24, 2002, incorporated herein in its entirety.

### FIELD OF THE INVENTION

The invention relates to nanoparticulate delivery systems for delivering a molecule of interest to the body. More particularly, the invention relates to a nanoparticle-based nucleic acid or protein vaccine comprising adjuvant and methods for delivering nucleic acid or protein to a target site using the nanoparticle-based vaccine of the invention.

### BACKGROUND OF THE INVENTION

Over the last twenty years, it has been established that the development of vaccines, including DNA vaccines, as particulates in the scale of micrometer or nanometer can help to improve the potency of the vaccines [O'Hagan, J. Pharm. Pharmacol. 49 (1997) 1-10; Singh, et al., Proc. Natl. Acad. Sci. USA 97 (2000) 811-816; and Kazzaz, et al., Control. Rel. 67 (2000) 347-356]. Previously, a novel nanoparticle-based DNA vaccine delivery system engineered from oil-in-waer (O/W) microemulsion precursors was developed by the present inventors. The microemulsions, formed at increased temperature (50-55° C.), were comprised of emulsifying wax (cetyl alcohol/polysorbate) as the oil phase and a cationic surfactant, cetyltrimethylammonium bromide CTAB. Upon simple cooling of these microemulsion precursors to room temperature in the same container, cationic nanoparticles (≦100 nm) were readily formed. Plasmid DNA was then coated on the surface of these pre-formed nanoparticles to form pDNA-coated nanoparticles. Both endosomolytic lipid, DOPE (dioleoyl phosphatidyl ethanolamine), and a potential dendritic cell-targeting ligand, mannan, were successfully incorporated in, or deposited on the surface of the nanoparticles to modify and/or improve the performance of the pDNA-coated nanoparticles both in vitro and in vivo. Immunization of mice with these pDNA-coated nanoparticles by subcutaneous injection, intradermal injection via a needle-free injection device, topical application on skin, or intranasal application led to enhanced immune responses to a model expressed antigen, β-galactosidase. For example, the antigen-specific total IgG titer in the sera of mice immunized with the pDNA-coated nanoparticles were enhanced by 16-200-fold over immunization with ‘naked’ pDNA alone by these routes of administration.

By definition, any material that aids the humoral and/or cellular immune responses to an antigen, but is itself immunologically inert, is referred to as an adjuvant. Adjuvants have been used to enhance the immune responses to antigens for about 70 years. During the last 70 years, many adjuvants have been developed, but few of them have been evaluated in clinical trials [R. Edelman, Vaccine Adjuvants, Rev. Infect. Dis. 2 (1980) 370-383]. One of the most studied and best-defined adjuvants is cholera toxin (CT). CT has mainly been used as an adjuvant for mucosal immunization by the intranasal or oral routes. Recently, Glenn et al. reported that CT, by co-administering with bovine serum albumin (BSA), can perform as an adjuvant to induce potent immune responses to BSA, when topically applied on shaved mouse skin. This so-called “transcutaneous immunization” has now proven to be a viable immunization modality in mice, sheep, cats, dogs, and even humans. Topical immunization with DNA vaccines on skin has also proven to be feasible [Tang et al., Nature 388 (1997) 729-730]. However, the potency of topical DNA immunization was found to be rather low.

The adjuvant effect of lipopolysaccharide (LPS) was first described as early as in 1956. The lipid A region of the LPS was found to be responsible for the adjuvanticity. Lipid A, which generally aids a Th1-type response, enhances immune responses primarily through its ability to activate antigen-presenting cells and to induce cytokine release. The first evidence that lipid A, an adjuvant conventionally used for protein (subunit)-based vaccines and other traditional vaccines, had an adjuvant effect with a DNA-based vaccine was reported by Sasaki et al. in 1997. Following this initial report, there were several other attempts to use lipid A as DNA vaccine adjuvant by different routes [Lodmell et al., Vaccine 18 (2000) 1059-1066; and Sasaki, et al., Infect. Immunol. 66 (1998) 823-826]. Another interesting property of lipid A is that it can also be used to enhance or complement the activity of antigen delivery vehicles such as ‘Alum’, liposomes [Fries, et al., Proc. Natl. Acad. Sci. USA 89 (1992) 358-362], and microparticles [Newman, et al., J. Control. Rel. 54 (1998) 49-59]. Recently, Wang et al. incorporated both pDNA and lipid A into poly(d,l-lactic-co-glycolic acid) (PLGA) microspheres for potential DNA vaccine delivery, although no in vivo results were reported [Wang, et al., J. Control. Rel. 57 (1999) 9-18].

The discovery that plasmid DNA vaccines can elicit both humoral and cellular immune responses has attracted much attention in the vaccine and immunology communities. However, after over a decade of intensive investigations, researchers have concluded that the potency of ‘naked’ pDNA vaccines is sub-optimal, especially in humans and non-human primates. Therefore, there exists a clear need to improve the effectiveness of DNA vaccines. To address this unmet need, the present inventors developed a novel nanoparticle-based vaccine delivery system comprising adjuvant.

As used herein the term “immunogen-containing nanoparticles” means nanoparticles that are coated with or admixed with an immunogen. The immunogen may be protein, peptide, or nucleic acid encoding an immunogeic protein or peptide. Nucleic acid may be DNA, RNA, oligonucleotides, and may be in either sense or antisense orientation.

### SUMMARY OF THE INVENTION

In one aspect of the invention there is provided a vaccine delivery system comprising a nanoparticle-based vaccine and adjuvant.

In another aspect of the invention there is provided a method of immunizing a patient comprising administering a nanoparticle-based vaccine delivery system comprising adjuvant.

### BRIEF DESCRIPTION OF THE DRAWINGS

FIG. 1 is a bar graph showing antigen-specific total IgG titer in sera to expressed β-galactosidase 45 days after non-invasive topical immunization on shaved mouse skin. Mice (n=6/group) were immunized with ‘naked’ pDNA (CMV-β-gal, 5 μg) mixed with 0 μg CT (DNA+0 CT), 10 μg CT (DNA+10 CT), or 100 μg CT (DNA+100 CT), or immunized with pDNA-coated nanoparticles mixed with either 0 μg CT (NPs+0 CT), 10 μg CT (NPs+10 CT), or 100 μg CT (NPs+100 CT) on day 0, 6, 21, and 35. Data reported are the geometric mean±standard deviation. A one-way ANOVA of the three mean serum IgG titer from mice immunized with ‘naked’ pDNA, with or without CT resulted in a p-value of 0.004, while similar analysis of mean serum IgG titer from mice immunized with DNA+0 CT, NPs+0 CT, NPs+10 CT, and NPs+100 CT resulted in a p-value of 0.016. (a) indicates that the result for the DNA+100 CT was significantly greater than that of DNA+10 CT and DNA+0 CT. (b) indicates that the result for the DNA+10 CT was significantly greater than that of DNA+0 CT. (c) indicates that the result for NPs+100 CT was significantly greater than that of the others. (d) indicates that the results from NPs+10 CT and NPs+0 CT were significantly greater than that of the DNA+0 CT, although NPs+10 CT and NPs+0 CT are not significantly different (p=0.28).

FIG. 2 is a bar graph showing in vitro proliferation of isolated splenocytes 45 days after topical immunization on shaved mouse skin. Mice (n=5-6/group) were immunized with ‘naked’ pDNA (CMV-β-gal, 5 μg) mixed with 0 μg CT (DNA+0 CT), 10 μg CT (DNA+10 CT), or 100 μg CT (DNA+100 CT), or immunized with pDNA-coated nanoparticles mixed with 0 μg CT (NPs+0 CT), 10 μg CT (NPs+10 CT), or 100 μg CT (NPs+100 CT) on day 0, 6, 21, and 35. The cell proliferation was reported as the % increase of the OD490 of the stimulated cells over their corresponding un-stimulated cells. Data reported are the mean±standard deviation (n=3). * indicates that the result from NPs+10 CT was significantly different from that of the NPs+10 OCT, NPs+0 CT, and Naïve. Splenocytes isolated from the naïve mice showed no response.

FIG. 3 is a bar graph showing antigen-specific total IgG titer in sera to expressed β-galactosidase 28 days after S.C immunization. Mice (n=6/group) were immunized with ‘naked’ pDNA (CMV-β-gal, 5 μg) mixed with 0 μg LA (DNA) or 50 μg LA (DNA+LA), or immunized with pDNA-coated nanoparticles mixed with either 0 μg LA (NPs) or 50 μg LA (NPs+LA) on day 0, 7, and 14. Data reported are the geometric mean±standard deviation of n=5-6. One-way ANOVA of the four mean serum IgG titer resulted in a p value of 0.0002. ** indicates that the result for NPs+LA was significantly different from that from the other groups. * indicates that the results for the NPs and DNA+LA were significantly different from that of the DNA. The results for NPs and DNA+LA were not significantly different (p=0.46).

### DETAILED DESCRIPTION OF THE INVENTION

Traditionally, vaccines have been comprised of live attenuated viruses or killed bacteria. However, DNA-based vaccines have attracted much attention recently. DNA-based vaccines may be safer than traditional vaccines and can elicit both humoral and cellular immune responses. In addition, DNA vaccines may be relatively stable, cost-effective for manufacture and storage, and may allow for potential simultaneous immunization against multiple antigens or pathogens. Further, CpG motifs on plasmid DNA have been shown to have an adjuvant effect. However, like other new generation vaccines, such as protein (subunit) vaccines and polysaccharide vaccines, DNA vaccines are relatively poorly immunogenic. Also, since the first proof-of-concept immunization with ‘naked’ pDNA, DNA vaccines have mainly been administered by intramuscular injection. Intramuscular injection of ‘naked’ pDNA vaccines has proven to be very effective in several different small animal models. However, recent results from human and non-human primate studies have been disappointing. Therefore, there is a clear need to improve the potency of DNA vaccines due to sup-optimal immune responses even when multi-milligram doses of pDNA are administered.

The present inventors have discovered that co-administration of immunogen containing nanoparticles and adjuvant (either simultaneous administration or administering adjuvant and nanoparticles within 24 hours of one another) results in enhanced immunogenicity of both nucleic acid based vaccines as well as protein or peptide based vaccines.

The nanoparticles used in the invention can be made to be cationic, anionic or neutral. For example, cationic nanoparticles can be made using a cationic surfactant such as cetyltrimethylammonium bromide (CTAB), anionic nanoparticles can be made using an anionic surfactant such as sodium dodecyl sulfate (SDS), and neutral nanoparticles can be made using a neutral surfactant such as polyoxyethylene 20 stearyl ether (Brij 78) or polyoxyethylene 20 sorbitan monooleate (polysorbate 80). Positively-charged or negatively charged antigens and adjuvants can then be coated on the surface of oppositely charged nanoparticles or may be admixed with the nanoparticles. For example, cationic nanoparticles can be coated with DNA, such as plasmid DNA, hepatitis B surface antigen, or oligonucleotides. Anionic nanoparticles can be coated with HIV proteins that are positively-charged such as Tat, gag p55, gag p24, or gp 120. Nucleic acids or proteins may be entrapped in neutral nanoparticles, or coated on the surface of neutral nanoparticles by hydrophobic interaction.

For the purposes of this invention, the adjuvants may be physically entrapped in the nanoparticles, coated or covalently attached on the surface of the nanoparticles, or co-mixed with a nanoparticle preparation. Alternatively, the adjuvant or mixture of adjuvants may be administered separately from the nanoparticle preparation.

Preferably, the nanoparticles are made from warm microemulsions by preparing a microemulsion from about 37-100° C. and cooling to form solid nanoparticles. It is preferred that the microemulsion is an oil-in-water microemulsion, but a water-in-oil-in-water microemulsion is also envisioned. The microemulsion may be prepared by melting an acceptable material between about 37° C.-100° C. to form an oil phase and then adding water to form a cloudy mixture of the melted oil in water. A surfactant is then added to form a clear or very slightly turbid microemulsion. Solid nanoparticles (cationic, anionic or neutral) are then formed directly from the warm microemulsion by simple cooling. Materials used to form the oil phase are solid at room temperature, but can be melted to form a liquid oil phase. Example of such materials are emulsifying wax, polyoxyethylene sorbitan fatty acid esters, polyoxyethylene alkyl ethers, polyoxyethylene stearates, phospholipids, fatty acids or fatty alcohols or their derivatives, or combinations thereof. Examples of surfactants used to form the warm microemulsions are positively-charged surfactants such as cetyltrimethylammonium bromide, negatively-charged surfactants such as sodium dodecyl sulfate, or neutral such as polyoxyethylene 20 stearyl ether (Brij 78) and polyoxyethylene 20 sorbitan monooleate (polysorbate 80). It is envisioned that any surfactant, regardless of charge, that promotes the formulation of a warm microemulsion may be used. It is preferred that the surfactant has a hydrophilic-lipophilic (HLB) value in the range of 6 to 20, and most preferred that the surfactant has an HLB value in the range of 8 to 18.

The immunogen-containing nanoparticles of the invention may be formed by coating nucleic acid e.g., plasmid DNA, mRNA, oligonucleotide, or protein or peptide fragments, and the like on the surface of pre-formed nanoparticles. Nucleic acids formulated with nanoparticles may range in size from small CpG oligonucleotides to larger fragments, e.g., plasmid DNA. The preferred CpG oligonucleotide has a molecular weight in the range of 1000 to 15000 daltons, and most preferred in the molecular weight range of about 2000 to 12,600 daltons. The preferred plasmid DNA has about 1000 base pairs to 15,000 base pairs, and most preferably between 1500 base pairs and 10,000 base pairs.

As discussed above, the nanoparticles may be engineered from warm oil/water microemulsion precursors by simple cooling at room temperature, for example. However, any suitable method of forming immunogen containing nanoparticles may be used. Preferably, the nanoparticles are in the size range of about 50 to about 500 nm, more preferably about 50 to about 300 nm, and most preferably about 100 nm. These immunogen containing nanoparticles are used together with an adjuvant, e.g., lipid A or cholera toxin, to immunize a patient.

It is understood that the skilled practitioner can vary the size and zeta potential of the particles as well as the final concentration of particles and adjuvant to be administered, depending, for example, on the size of the animal to whom the particles are being delivered. Zeta-potential is defined as the surface charge at the nanoparticle surface. The particle size and zeta-potential (surface charge) of solid nanoparticles made directly from warm microemulsions may be easily characterized. The particle sizes of engineered nanoparticles can be measured using N4 Plus Sub-Micron Particle Sizer (Coulter Corporation, Miami, Fla.) using photon correlation spectroscopy (PCS). The zeta-potential of the nanoparticles can be measured using an electrophoretic light scattering instrument, e.g., Zeta Sizer 2000 (Malvern Instruments, Inc., Southborough, Mass.) using electrophoretic light scattering and is most commonly reported in millivolts (mV). Cationic nanoparticles, made with a positively-charged surfactant usually have a zeta-potential in the range of about +1 to about +100 mV, with the most preferred range of about +5 mV to about +80 mV. Anionic nanoparticles, made with a negatively-charged surfactant usually have a zeta-potential in the range of about −1 to about −100 mV, with the most preferred range of about −5 mV to about −80 mV.

It is envisioned that a number of different adjuvants can be entrapped in the nanoparticles, coated on the surface surface, or co-mixed with a nanoparticle preparation. Nonlimiting examples of adjuvants that may be used in the nanoparticle vaccine delivery system of the invention are cytokines such as Interleukin-2 (IL-2) and IL-12, saponins, muramyl-di-peptides (MDP) or derivatives, CpG oligonucleotides, lipopolysaccharides or derivatives, cholera toxin or its subunits, or adjuvants which are known as ligands for the toll-like receptors such as tri-acyl lipopeptides, lipoteichoic acid, glycolipids, lipopolysaccharides, heat-shock proteins, single or double-stranded RNA, and synthetic compounds such as imidazoquinoline. Toll-like receptors (TLR) are part of the innate immune system that recognize specific compounds (also known as ligands) present in microorganisms. Activation of TLRs by these ligands results in the induction of inflammatory responses and the production of antigen-specific adaptive immunity. It will also be appreciated by those skilled in the art that many adjuvants have either charge (CpG oligonucleotides, lipoteichoic acid, double-stranded RNA) that make them amenable for surface coating on oppositely-charged nanoparticles or have lipophilic properties that allow them to be easily entrapped in nanoparticles made from oil-in-water microemulsion precursors.

Effective vaccines against various pathogens may require more of a cellular immune response or a humoral immune response, or a balance of both a cellular and humoral immune response. Thus, the preferred adjuvant or combination of adjuvants will bias the immune response to that needed for protection or a therapeutic response against a particular pathogen.

In the production of a nanoparticle based vaccine delivery system, the final concentration of nanoparticles, antigen, and adjuvant has an impact on the effectiveness of the vaccine. The preferred nanoparticle concentration for administration is about 10 to about 10,000 ug/ml, with the most preferred nanoparticle concentration of about 100 to about 2000 ug/ml. The preferred antigen concentration is about 1 to about 1000 ug/ml, with the most preferred antigen concentration of about 1 to about 500 ug/ml. The preferred adjuvant concentration for administration is about 1 to about 5000 ug/ml, with the most preferred adjuvant concentration of about 1 to about 2000 ug/ml. However, the most effective vaccine against a particular pathogen may require titration of the nanoparticle, adjuvant, and antigen concentrations for administration.

There are many suitable routes for administering an effective vaccine to a patient, such as an animal or particularly, a warm-blooded animal. In recent years, mucosal routes have attracted a great deal of interest since this is the mechanism that most pathogens invade the body. Mucosal routes of immunization include, but are not limited to, nasal, vaginal, rectal, and buccal. Non-invasive methods of administration have also been sought since they may afford immunization without the use of needles. Non-invasive routes of administration include, topical on the skin, nasal, vaginal, rectal, and buccal.

The parenterally routes of administration such as intramuscular, subcutaneous, and intradermal have also been shown to be effective routes of immunization. The preferred routes of administration for this invention include the mucosal routes, routes that are non-invasive, and the parenteral routes.

Non-invasive topical immunization with vaccines on skin is attractive since the skin is readily accessible, and known to be one of the largest organs of the immune system. The skin is rich in the potent antigen presenting cells (APC) such as Langerhan's cell (LCs) and Dendritic cells (DCs). It is also well equipped with other necessary immune cells and cytokines. Topical immunization, due to its needleless nature, may be more cost-effective and have increased patient compliance, and therefore, allows for widespread vaccination. Although the feasibility of non-invasive topical DNA immunization was established as early as 1997, its very low potency has limited further applications. Therefore, methods to improve its potency are still needed.

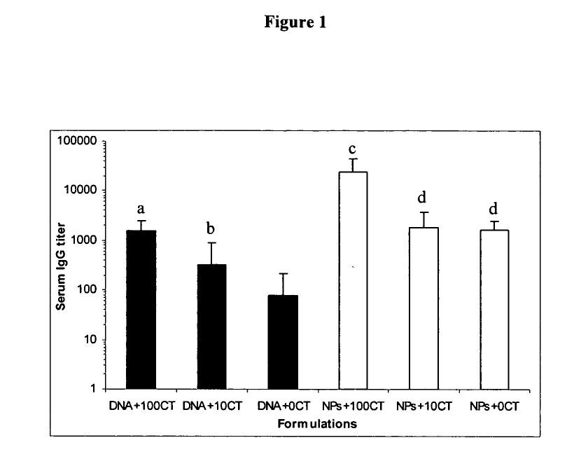

As shown in FIG. 1, the co-administration of adjuvant, e.g., cholera toxin with ‘naked’ pDNA leads to a significant enhancement in specific total IgG titer in sera to an expressed antigen, (β-galactosidase in FIG. 1), compared to immunization without adjuvant. For example, the total serum IgG titer from mice immunized with the pDNA with cholera toxin (100 μg), and pDNA with cholera toxin (10 μg) were 20-fold (p=0.004) and 4-fold (p=0.02) greater, respectively, than that from the mice immunized with ‘naked’ pDNA alone without cholera toxin.

Moreover, the IFN-γ released from splenocytes isolated from mice immunized with pDNA with cholera toxin was significantly higher than that from mice immunized without cholera toxin (Table 1). Mice were immunized topically on shaved skin with either ‘naked’ pDNA mixed with 0 μg CT (DNA+0 CT), 10 μg CT (DNA+10 CT), or 100 μg CT (DNA+100 CT) or with pDNA-coated nanoparticles mixed with 0 μg CT (NPs+0 CT), 10 μg CT (NPs+10 CT), or 100 μg CT (NPs+100 CT). Naive mice were not treated. Splenocyte preparation and cytokine release studies were completed as described above. The results are shown below in Table 1.

TABLE 1
In vitro cytokine release profiles from isolated splenocytes.

| | IFN-γ (pg/mL) | IL-4 (pg/mL) |
|---|---|---|
| DNA + 100CT | 722.6 ± 51.3* | 45.5 + 0.6 |
| DNA + 10CT | 422.3 ± 67.3* | 33.4 ± 6.7 |
| DNA + 0CT | 216.9 ± 52.2 | 51.5 ± 14.8 |
| NPs + 100CT | 224.9 ± 77.8 | 33.6 ± 16.6 |
| NPs + 10CT | 640.6 ± 35.5** | 51.8 ± 6.6*** |
| NPs + 0CT | 342.4 ± 133.5 | 24.8 ± 7.6 |
| Naïve | 194.1 ± 2.5 | 32.3 ± 5.4 |

Data are the mean ± standard deviation (n = 3).
*indicates that, for IFN-γ, the results for DNA + 100CT and DNA + 10CT were significantly different from that for the DNA + 0CT and naive.
**indicates that, for IFN-γ, the result for NPs + 10CT was different from that for the NPs + 100CT, NPs + 0CT, and naïve.
***indicates that, for IL-4, the result for NPs + 10CT was different from that for the NPs + 100CT, NPs + 0CT, and naïve.

These enhancements in IFN-γ release were also dependent on the cholera toxin dose. These results, in combination with the observation that the IL-4 release was not increased by the co-administration of the cholera toxin, demonstrated that cholera toxin performs as an adjuvant for non-invasive topical DNA immunization, and that both enhanced antibody response and more Th1-biased T cell responses are elicited. Topical immunization with the pDNA-coated nanoparticles, compared to immunization with ‘naked’ pDNA alone, enhanced the specific total IgG titer in sera by 21-fold (p=0.002), to a level that was comparable to immunization with ‘naked’ pDNA with cholera toxin (100 μg) (FIG. 1). This enhancement with pDNA-coated nanoparticles was similar to that observed in previous studies by the inventors [Cui, et al., J. Control. Rel. 81 (2002) 173-184]. Also, as shown in FIG. 1, the specific IgG titer in sera was enhanced by 14-fold (p=0.02) when mice were immunized with the pDNA-coated nanoparticles with 100 μg cholera toxin, as compared to immunization with the pDNA-coated nanoparticles without CT. The specific total IgG titer from the mice topically immunized with pDNA-coated nanoparticles with 100 μg of cholera toxin was over 300-fold higher than that from mice immunized with ‘naked’ pDNA alone, strongly indicating an unexpected synergistic effect from the nanoparticles and cholera toxin in inducing antibody production.

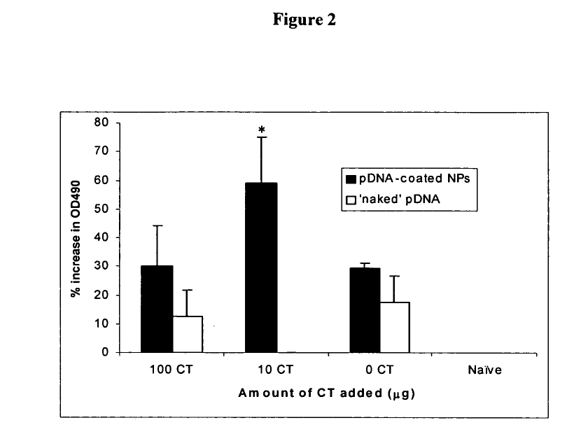

Shown in Table 1 and FIG. 2 are the results of in vitro cytokine release and proliferation by the isolated splenocytes. Again, co-administration of the pDNA-coated nanoparticles with cholera toxin helped to enhance both cytokine release and splenocyte proliferation, although the enhancement was not directly related to the dose of cholera toxin. In fact, pDNA-coated nanoparticles with 10 μg of cholera toxin led to enhanced IFN-γ release, IL-4 release, and splenocyte proliferation, while immunization with 100 μg of cholera toxin did not show any apparent effect. These results suggested that the amount of cholera toxin co-administered with pDNA-coated nanoparticles can be further optimized to obtain optimal immune responses. However, cholera toxin co-administrated with either ‘naked’ pDNA alone or with pDNA-coated nanoparticles boosted the production of specific antibody (IgG), increased the release of Th1-type cytokine (IFN-γ) from isolated splenocytes, and enhanced splenocyte proliferation.

The exact mechanism(s) behind the observed adjuvant effect are currently unknown. Using skin transplantation experiments, Fan et al. concluded that pDNA vaccines may enter the skin through the hair follicles [Nat. Biotech. 17 (1999) 870-872]. Therefore, one possibility for the adjuvant effect, such as that observed with cholera toxin may be that adjuvant can enhance the access of pDNA via the hair follicles. Also, it is possible that the adjuvant may be a signal to produce an inflammatory response, and thereby, cause antigen presenting cells like DCs to migrate to the hair follicle sites.

It is well known that non-invasive DNA immunization on skin with ‘naked’ pDNA alone is very inefficient in inducing immune responses. In six independent immunization studies in Balb/C mice by topical application of ‘naked’ pDNA alone (4-100 μg) on skin, average specific total IgG titer with geometric means below or close to 100 were observed, with most of the mice being non-responders. This observation agreed with other reports in the literature. In contrast, after topical immunization with pDNA-coated nanoparticles with cholera toxin (100 μg) on shaved mouse skin, specific total IgG titer with a geometric mean of 24,000 was obtained, strongly indicating that a therapeutically relevant level of serum IgG is achievable. Due to its strong toxicity, administration of cholera toxin by the parenteral, oral, or nasal routes was precluded. However, this toxicity issue can be avoided by administering cholera toxin non-invasively on skin.

Effect of Co-Administration of Lipid A on DNA Immunization by Subcutaneous Injection

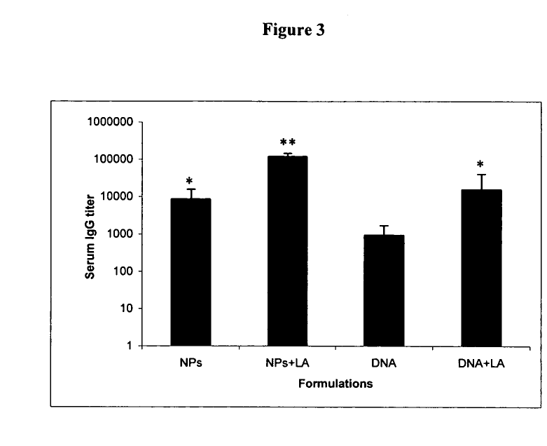

Shown in FIG. 3 are the specific total IgG titer in the sera of mice immunized with either ‘naked’ pDNA alone or pDNA-coated nanoparticles, with or without lipid A (50 μg) by subcutaneous injection. Immunization with pDNA-coated nanoparticles led to more than 16-fold enhancement in total serum IgG titer over immunization with ‘naked’ pDNA alone (p=0.038), which agreed well with previous reports. Co-administration of lipid A with ‘naked’ pDNA also resulted in close to 16-fold enhancement in serum total IgG titer (p=0.029) over immunization with pDNA alone. Specifically, the total IgG titer from mice immunized with pDNA-coated nanoparticles and lipid A was 16-fold (p<0.05) higher than that from mice immunized with pDNA-coated nanoparticles alone, and over 250-fold (p=0.0002) greater than that from mice immunized with ‘naked’ pDNA alone. These results strongly demonstrate that pDNA-coated nanoparticles and lipid A, when administered together, synergistically enhance the resulting antibody responses.

Table 2 shows the in vitro cytokine release from isolated splenocytes after stimulation with β-galactosidase protein. Mice were immunized subcutaneously with either ‘naked’ pDNA mixed with 0 μg LA (DNA) or 50 μg LA (DNA+LA) or with pDNA-nanoparticles mixed with 0 μg LA (NPs) or 50 μg LA (NPs+LA). Naive mice were not treated. Splenocyte preparation and cytokine release studies were completed as mentioned in the Materials and Methods section. Data reported are the mean±standard deviation (n=3). A one-way ANOVA revealed no significant different between all the IL-4 data (p=0.31). However, for the IFN-γ data, a p-value of 0.013 was obtained after one-way ANOVA analysis. * indicates that the IFN-γ result for NPs+LA was statistically different from the IFN-γ results for all other group. ** indicates that the INF-γ level for DNA+LA was statistically different from that for DNA. Also, except for the DNA, the IFN-γ concentrations from all other immunized groups were statistically different from the naïve group.

TABLE 2
In vitro cytokine release profiles from isolated splenocytes.

| | IFN-γ (pg/mL) | IL-4 (pg/mL) |
|---|---|---|
| Naïve | 1155 ± 70 | 60 ± 7 |
| NPs | 2008 ± 395 | 73 ± 2 |
| NPs + LA | 3159 ± 230* | 79 ± 4 |
| DNA | 1025 ± 50 | 62 ± 10 |
| DNA + LA | 2056 ± 537** | 83 ± 12 |

A one-way ANOVA analysis showed no statistical difference in the IL-4 levels among all groups tested (p=0.31). However, both immunization with the pDNA-coated nanoparticles and immunization with ‘naked’ pDNA with lipid A led to significantly enhanced IFN-γ release, compared to immunization with ‘naked’ pDNA alone. Again, splenocytes isolated from mice immunized with pDNA-coated nanoparticles with lipid A released the highest amount of IFN-γ after stimulation. Co-administration of lipid A also led to more positive cases of proliferation and greater extent of proliferation of isolated splenocytes than immunization without lipid A for both ‘naked’ pDNA and pDNA-coated nanoparticles (Table 3).

TABLE 3
In vitro proliferation of isolated splenocytes.

| | Positive cases of | Extent of |
|---|---|---|
| | proliferation | proliferation |
| Naïve | 0 (3) | N/A |
| NPs | 1 (3) | 43% |
| NPs + LA | 3 (3) | 44-145% |
| DNA | 1 (3) | 29% |
| DNA + LA | 3 (3) | 8-49% |

Mice were immunized subcutaneously with either ‘naked’ pDNA mixed with 0 μg LA (DNA) or 50 μg LA (DNA+LA) or with pDNA-coated nanoparticles mixed with 0 μg LA (NPs) or 50 μg LA (NPs+LA) on day 0, 7, and 14. Naïve mice were not treated. On day 28, the mice were sacrificed and their spleens were removed. Two spleens from the same group were pool together so that each treatment had 3 splenocyte preparations. Isolated splenocytes (5×10⁶/well) were incubated with either 0 or 3.3 μg/well of β-galactosidase protein for 94 h. Cell proliferation results were reported as the % increase of the OD490 of the stimulated cells over their corresponding un-stimulated cells.

Earlier studies with lipid A demonstrated that its adjuvant activity is related to its potential to activate macrophages and its ability to induce IFN-γ and IL-2, both known to be essential for the induction of Th1 type cell-mediated immune responses. In 1997, Sasaki et al. studied the effect of co-administration of monophosphoryl lipid A with a DNA vaccine encoding HIV-1 env and rev genes on the resulting immune responses and hypothesized that the lipid A could help to further boost the Th1-type cytokine release [Infect. Immunol. 65 (1997) 3520-3528]. The authors reported that the serum from mice immunized by intramuscular injection with the lipid A preparation revealed 60 to 500-fold higher HIV-1 specific IgG titer than the sera from mice immunized without lipid A. HIV-1 specific IgG subclass analysis showed that lipid A tends to facilitate IgG2a production, suggesting enhancement of a predominant Th1 type response [Saiki et al, 1997]. These observations agree well with those obtained by the present inventors. The specific IgG titer in the sera of the mice immunized with ‘naked’ pDNA with lipid A was over 16-fold higher than that in the mice immunized without lipid A. Also, in vitro cytokine release studies revealed that the enhancement was biased towards a Th1 type response.

Lipid A has been shown to have adjuvant activity when used alone, or in combination with other immunostimulants and delivery systems [Fries, et al. Proc. Natl. Acad. Sci. USA 89 (1992) 358-362; Newman, et al, J. Control. Rel. 54 (1998) 49-59; and Baldridge, et al., Methods 19 (1999) 103-107]. For example, Newman et al. reported that following subcutaneous immunization, incorporation of monophosphoryl lipid A in ovalbumin (OVA)-loaded PLGA microspheres resulted in increased production of IFN-γ, when compared to OVA-loaded PLGA microspheres without the incorporation of lipid A. Also, immunization with OVA-loaded PLGA microspheres without incorporated lipid A resulted in increased IFN-γ production compared to immunization with OVA alone. In the present invention, a DNA vaccine is used with nanoparticles, and surprisingly, the results agree well with the observations by Newman et al. using a protein-based vaccine.

The methods of the present invention demonstrate that immunization with nucleic acid-coated nanoparticles leads to enhanced Th1 type cytokine release compared to immunization with ‘naked’ nucleic acid, i.e., pDNA, alone. Moreover, co-administration of lipid A with the nucleic acid-coated nanoparticles further enhances IFN-γ release over immunization with the nucleic acid-coated nanoparticles alone. By intramuscular and subcutaneous injection, DNA vaccines are known to favor the production of Th1 type responses, which are important for the induction of cell-mediated immune responses. One of the reasons for the lack of effective vaccines for HIV, malaria and tuberculosis is that most of the current vaccines fail to induce cellular immune responses, which are thought to be equally as critical as inducing neutralizing antibodies for successful prevention of these pathogens. Nucleic acid vaccines are thought to be promising for the development of effective vaccines for these pathogens. Therefore, the strategy of combining lipid A with a nanoparticle-based delivery system has potential to elicit both enhanced antibody production and Th1-biased immune responses.

The toxicity associated with lipid A may be avoided by using the detoxified monophosphoryl lipid A (MPL®), which has proven to be as effective as the original lipid A in enhancing immune responses, while at the same time being less toxic than lipid A (100 to 1000-fold).

The methods of the present invention demonstrate for the first time that cholera toxin performs as an effective adjuvant in non-invasive topical nucleic acid immunization. The use of adjuvant such as cholera toxin results in enhanced antibody production and more Th1-biased immune responses. In addition, co-administration of a nanoparticle-based nucleic acid vaccine delivery system with known adjuvants, for example, either cholera toxin or lipid A, and in particular, detoxified lipid A, synergistically enhances the resulting immune responses obtained from a nucleic acid vaccine. For example, topical non-invasive immunization of mice with the nucleic acid-coated nanoparticles with about 100 μg of CT led to over 300-fold increase in antigen specific IgG titer than immunization with ‘naked’ nucleic acid alone. Also, an over a 250-fold enhancement in IgG titer was observed when mice were subcutaneously immunized with the nucleic acid-coated nanoparticles with 50 μg of lipid A, compared to immunization with ‘naked’ nucleic acid alone. The results demonstrate that the combination of known adjuvants with the delivery system is an effective method of immunizing against disease.

### EXAMPLES

### Example 1

### Engineering of Plasmid DNA-Coated Nanopaticles

Plasmid DNA-coated nanoparticles were prepared by coating CMV-β-gal (pDNA) on pre-formed cationic nanoparticles as previously described [Cui et al., Pharm. Res. 19 (2002) 939-946; and Cui, J. Control. Rel. 81 (2002) 173-184]. Briefly, emulsifying wax (2 mg/mL) was melted at 55° C. Seven hundred (700) μL of water was added into the melted wax and stirred until a homogenous milky suspension was obtained. Then, 0.3 mL of CTAB solution (50 mM) was added into the homogenate while stirring to obtain a clear microemulsion. Nanoparticles were engineered by simple and direct cooling of this warm microemulsion to room temperature in the same container. For the incorporation of endosomolytic agent, 100 μg of DOPE (final 5% w/w) was mixed with the emulsifying wax (2 mg/mL) prior to microemulsion preparation. Chol-mannan, dissolved in hot water (5 mg/mL), was deposited on the surface of the nanoparticles by mixing 1 mL of the pre-formed nanoparticle suspension (2 mg/mL) with 250 μg of chol-mannan and stirred at room temperature overnight. Free CTAB and chol-mannan were removed by passing the nanoparticle suspension through a Sephadex G-75 column (14×65 mm) using 10% lactose as the mobile phase. Plasmid DNA (CMV-β-gal) was coated on the surface of these pre-formed cationic nanoparticles by gently mixing 1 mL of the purified and filtered nanoparticles in suspension with pDNA to obtain a final pDNA concentration of 50 μg/mL. This system was allowed to remain for at least 30 minutes at room temperature for complete adsorption of pDNA on the surface of the nanoparticles before further use. The particle sizes and zeta potentials of engineered nanoparticles, before and after pDNA coating, were measured using N4 Plus Sub-Micron Particle Sizer (Coulter Corporation, Miami, Fla.) and Zeta Sizer 2000 (Malvern Instruments, Inc., Southborough, Mass.), respectively.

### Example 2

### Immunization of Mice

Ten to twelve week old female mice (Balb/C) from Harlan Sprague-Dawley Laboratories were used for all animal studies. Two independent mouse studies were completed. Mice were immunized either by subcutaneous injection or by non-invasive topical application on the skin. SC immunization was performed as previously described by Cui, et al., Pharm. Res. 19 (2002) 939-946 with modification. Briefly, on day 0, day 7, and day 14, mice (n=6/group) were immunized with either ‘naked’ pDNA alone (CMV-β-gal, 5 μg) or pDNA (5 μg)-coated nanoparticles, mixed with 0 or 50 μg of lipid A prepared as an aqueous solution in 0.5% (v/v) triethanolamine in water. Mice were anesthetized using pentobarbital (i.p.) prior to each immunization. A volume of 150 μl of each formulation (in 10% lactose) was injected using an Insulin Syringe with MICRO-FINE® IV Needle by Becton Dickinson and Company (Franklin Lakes, N.J.) on one site on the back. Naïve mice (n=6) were not treated. On day 28, the mice were anesthetized and bled by cardiac puncture. Sera were separated and stored as previously described by Cui, et al., , Pharm. Res. 19 (2002) 939-946. Spleens from every mouse were also collected on day 28.

Topical immunization on mouse skin was completed as previously described by Cui, et al., J. Control. Rel. 81 (2002) 173-184 with modification. Mice (n=6/group) were immunized with either ‘naked’ pDNA or pDNA-coated nanoparticles, mixed with 0, 10, or 100 μg of cholera toxin, on day 0, 6, 21, and 35 with a pDNA dose of 5 μg. Again, mice were anesthetized using pentobarbital (i.p.) prior to each immunization. The hair covering the back of the mouse was shaved with an A5® Single-Speed Clipper (Oster Professional Products, McMinnville, Tenn.). The skin was wiped with an alcohol swab, allowed to air dry for 5 min, and 120 μL of each formulation was dripped and subsequently spread with a pipette tip onto the skin covering an area of about 2 cm². Care was taken to disperse the solution over the skin without applying pressure to the skin. On day 45, the mice were anesthetized, and the blood and spleens were collected and treated as described above. One group of naïve mice was not treated and used as a negative control.

### Determination of Antibody Titer

β-galactosidase-specific serum IgG titer was quantified using ELISA. Briefly, Costar® high binding 96-well assay plates were coated with 50 μL of β-galactosidase protein (8 μg/mL) overnight at 4° C. The plates were then blocked for 1 hour at 37° C with 4% bovine serum albumin (BSA)/4% NGS (Sigma) solution (100 μL/well) made in 1×PBS/Tween 20 (Scytek). Mouse serum (50 μL/well, serial diluted and starting at 1:10 [for topical] or 1:64 [for SC] in 4% BSA/4% NGS/PBS/Tween 20) was incubated for 2 hours at 37° C. After washing three times with PBS/Tween 20 buffer, anti-mouse IgG HRP F(ab′)₂ fragment from sheep (diluted 1:3,000 in 1% BSA) was added (50 μL/well) and incubated for 1 hour at 37° C. Plates were washed three times with PBS/Tween 20 buffer. Finally, the samples were developed with 100 μL TMB substrate for 30 min at room temperature and then stopped with 50 μL of 0.2 M H₂SO₄. The optical density of each well was measured using a Universal Microplate Reader (Bio-Tek Instruments, Inc., Winooski, VM) at 450 nm.

### In Vitro Cytokine Release and Splenocyte Proliferation

Splenocyte preparation, cytokine release and splenocyte proliferation assays were performed as previously described by Cui, et al., J. Control. Rel. 81 (2002) 173-184. Spleens from two mice in the same group were pooled together (i.e., N=3 per treatment) and placed into 5 mL of HBSS (Hank's Balanced Salt Solution) (1×) in a Stomacher Bag 400 from Fisher Scientific (Pittsburgh, Pa.). The spleens were homogenized at high speed for 60 seconds using a Stomacker Homogenizer. Cell suspensions were then transferred into 15 mL Falcon tube and filled to 15 mL with 1×ACK buffer (156 mM of NH₄Cl, 10 mM of KHCO₃, and 100 μM of EDTA) for red blood cell lysis. After 5-8 min at room temperature, the suspension was spun down at 1,500 rpm for 7 minutes at 4° C. After pouring off the supernatant, the cell pellet was re-suspended in 15 mL 1×HBSS. The suspension was then spun down at 1,500 rpm for 7 min at 4° C. After washing with 15 mL of RPMI-1640 (BioWhittaker, Walkersville, Md.) supplemented with 10% fetal bovine serum (FBS) (Sigma, St. Louis, Mo.) and 0.05 mg/mL of gentamycin (Gibco BRL), the cells were re-suspended in RPMI 1640 media (2 mL total or 1 mL for each spleen in the pool).

For in vitro cytokine release, isolated splenocytes (5×10⁶/well) were seeded into a 48-well plate (Costar), and stimulated with 0 or 3.3 μg/well of β-galactosidase (Spectrum) for 48 hours at 37° C. Cytokines (IL-4 and IFN-γ) in the supernatant were quantified using ELISA kits from Endogen.

A CellTiter 96® Aqueous non-radioactive cell proliferation assay kit was used to determine the isolated splenocyte proliferation. Similarly, isolated splenocytes (5×10⁶/well) were seeded into a 48-well plate (Costar), and stimulated with 0 or 3.3 μg/well of β-galactosidase (Spectrum). After incubation at 37° C. with 5% CO₂ for 94 hours, 60 μL of the combined 3-(4,5-dimethylthiazol-2-yl)-5-(3-carboxymethoxylphenyl)-2-(4-sulfophenyl)-2H-tetrazolium/phenazine methosulfate (MTS/PMS) solution (Promega) was pipetted into each well (20 μL/100 μL of cells in medium). After an additional one hour of incubation at 37° C. with 5% CO₂, the absorbance at 490 nm was measured using a Universal Microplate Reader. The cell proliferation was reported as the % increase of the OD₄₉₀ of the stimulated cells (3.3 μg/well) over the OD₄₉₀ of un-stimulated cells (0 μg/well) (i.e., 100× (OD490_{stimulated}−OD490_{un-stimulated})/OD490_{un-stimulated).}

### Statistical Analyses

Except where mentioned, all statistical analyses were completed using a one-way analysis of variances (ANOVA) followed by pair-wise comparisons with Fisher's protected least significant difference procedure (PLSD). A p-value of ≦0.05 was considered to be statistically significant.

Plasmid containing a CMV promoter with a β-galactosidase reporter gene (CMV-β-gal) was a gift from Valentis, Inc. (The Woodlands, Tex.). The plasmid had endotoxin levels <0.1 EU/mg. Emulsifying wax (N.F. grade) was purchased from Spectrum Quality Products, Inc. (New Brunswick, N.J.). Cetyltrimethylammonium bromide (CTAB), β-galactosidase, normal goat serum (NGS), bovine serum albumin (BSA), triethanolamine (TEA), and Sephadex G-75 were from Sigma Chemical Co. (St. Louis, Mo.). PBS/Tween 20 buffer (20×) was from Scyteck Laboratories (Logan, Utah). Anti-mouse IgG peroxidase-linked species specific F(ab′)₂ fragment (from sheep) was purchased from Amersham Pharmacia Biotech Inc. (Piscataway, N.J.). Tetramethylbenzidine (TMB) soluble reagent was from Pierce (Rockford, Ill.). Dioleoyl phosphatidylethanolamine (DOPE) was purchased from Avanti Polar Lipids, Inc. (Alabaster, Ala.). {N-[2-(Chloesterylcarboxyamino)ethyl]carbamoylmethyl}mannan (chol-mannan) was purchased from Dojindo Molecular Technologies, Inc. (Gaithersburg, Md.). Lipid A from Salmonella Minnesota R595 (Re) lipopolysaccharide and cholera toxin from Vibrio cholera Inaba 569B were purchased from List Biological Laboratories, Inc. (Campbell, Calif.). Mouse Interleukin-4 (IL-4) and Interferon-γ (IFN-γ) ELISA Kits were from Pierce-Endogen, Inc. (Woburn, Mass.). CellTiter 96® Aqueous non-radioactive cell proliferation assay kit was purchased from Promega (Madison, Wis.).

What is claimed is:

1. A vaccine delivery system comprising adjuvant and a plurality of nanoparticles comprising immunogenic antigen or nucleic acid encoding an immunogenic antigen.

2. The vaccine delivery system of claim 1 wherein the nanoparticles are cationic.

3. The vaccine delivery system of claim 1 wherein the nanoparticles are anionic.

4. The vaccine delivery system of claim 1 wherein the nanoparticles are neutral.

5. The vaccine delivery system of claim 1 wherein the nanoparticles comprise an anionic surfactant.

6. The vaccine delivery system of claim 1 wherein the nanoparticles comprise a cationic surfactant.

7. The vaccine delivery system of claim 1 wherein the nanoparticles comprise a neutral surfactant.

8. The vaccine delivery system of claim 1 wherein the nanoparticles are coated or admixed with nucleic acid encoding an immunogenic polypeptide.

9. The vaccine delivery system of claim 1 wherein the immunogenic antigen is a polypeptide or peptide.

10. The vaccine of claim 1 wherein the adjuvant is selected from the group consisting of cholera toxin, lipid A, and monophosphoryl lipid A.

11. The vaccine of claim 1 wherein the adjuvant is cholera toxin.

12. The vaccine of claim 1 wherein the adjuvant is lipid A or monophosphoryl lipid A.

13. The vaccine of claim 1 wherein the nucleic acid is DNA.

14. The vaccine of claim 1 wherein the nucleic acid is an oligonucleotide.

15. A method of immunizing an animal comprising administering to the animal a vaccine delivery system comprising adjuvant and a plurality of nanoparticles comprising immunogenic antigen or nucleic acid encoding immunogenic antigen.

16. The method of claim 15 wherein the adjuvant and vaccine are administered simultaneously.

17. The method of claim 15 wherein the adjuvant is administered within 24 hours of administering the nanoparticles.

18. The method of claim 15 wherein the vaccine delivery system is administered via a non-invasive, parenteral, or mucosal route.

19. The method of claim 15 wherein the vaccine delivery system is administered topically.

20. The method of claim 15 wherein the vaccine delivery system is delivered subcutaneously.

21. The method of claim 15 wherein the adjuvant is lipid A or monophosphoryl lipid A.

22. The method of claim 15 wherein the adjuvant is cholera toxin.

23. The method of claim 15 wherein the nucleic acid is DNA.

24. The method of claim 15 wherein the nucleic acid is RNA.

25. The method of claim 15 wherein the nucleic acid is an oligonucleotide.

26. A method for enhancing a Th1 response in a patient comprising administering a vaccine delivery system comprising adjuvant and a plurality of nanoparticles comprising an immunogenic antigen or nucleic acid encoding an immunogenic antigen to the patient.
